(19) United States
(12) Patent Application Publication
(10) Pub. No.: US 2017/0218397 A1
(43) Pub. Date: Aug. 3, 2017

(54) CONDITIONAL CYTOTOXIC GENE THERAPY VECTOR FOR SELECTABLE STEM CELL MODIFICATION FOR ANTI HIV GENE THERAPY

(71) Applicant: Texas Tech University System, Lubbock, TX (US)

(72) Inventors: Himanshu Garg, El Paso, TX (US); Anjali Joshi, El Paso, TX (US)

(21) Appl. No.: 15/329,318

(22) PCT Filed: Jul. 20, 2015
(86) PCT No.: PCT/US15/41146

§ 371 Date: Jan. 26, 2017

Related U.S. Application Data
(60) Provisional application No. 62/030,783, filed on Jul. 30, 2014.
(60) Provisional application No. 62/194,506, filed on Jul. 20, 2015.

(51) Int. Cl. C12N 15/86; C07K 14/005; A61K 38/45; A61K 31/519; C12N 9/12; A61K 48/00

(52) U.S. Cl. CPC C12N 15/86; C12N 9/1211; C12Y 207/01021; A61K 48/0058; A61K 38/45; A61K 31/519; A61K 48/0091; C07K 14/005; C12N 2740/15043; C12N 2740/15052; C12N 2740/16022

(57) ABSTRACT

A method, system, and apparatus for treating a patient with HIV. A vector can be modified from a thymidine kinase gene. The modified vector is expressed in the presence of tat RNA. The modified vector is then package and delivered to HIV-infected cells. The replication of HIV is inhibited by eliminating infected cells in the presence of Ganciclovir. Modified cells are then selected utilizing transient tat RNA transfection and GFP expression. Vector-modified stem cells are then selected for transplantation back into the patient, thereby producing a normal immune system in the patient when the modified vector remains dormant in the absence of HIV tat.

### STATEMENT OF GOVERNMENT RIGHTS

This invention was made with government support under contract No. 1R03A1095040-01A1 awarded by the National Institute of Health. The government has certain rights in the invention.

### TECHNICAL FIELD

Embodiments generally relate to cytotoxic gene therapy for selectable stem cell modification thus providing anti-HIV gene therapy. Embodiments further relate to the selective cytotoxic elimination of infected cells and suppression of HIV replication.

### BACKGROUND

The rapidly mutating nature of HIV (Human Immunodeficiency Virus) and the adverse effects associated with routine drug therapy have necessitated the development of alternative therapeutic interventions. One of the obvious alternatives to chemotherapy is gene therapy. Advances in human CD34+ stem cell transplantation have prompted a search for new and potent gene therapy targets for suppression of HIV replication.

Furthermore, there is always a need for novel anti-HIV drugs with unique mechanisms of action that can be harnessed as salvage therapy for terminally ill HIV patients. Small hairpin RNAs (shRNAs) targeting viral or host genes have been the most commonly studied non-conventional gene therapy approach for controlling HIV infection. shRNA-mediated therapy, however, comes with the caveat that escape mutants arise rapidly due to the ease with which an shRNA is rendered nonfunctional with small sequence changes. Moreover, long-term shRNA therapy results in cell toxicity from competition with the host micro RNA processing machinery.

Three important factors have hindered the development of HIV gene therapy. These include: 1) development of resistance against the gene therapy vector; 2) selection of CD34+ stem cells modified with the vector without altering the functionality “stemness” of the cells; and 3) having a safe and effective vector to combat HIV infection.

Therefore, a need exists for an improved HIV treatment utilizing a modified vector that selectively targets infected cells.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the disclosed embodiments to provide a method and system for treating a patient with HIV.

It is another aspect of the disclosed embodiments to provide a method and system for a vector for treating a patient with HIV.

It is yet another aspect of the disclosed embodiments to provide an enhanced method and system for a unique vector for modification of CD34+ Hematopoietic stem cells for anti-HIV gene therapy.

The aforementioned aspects and other objectives and advantages can now be achieved as described herein. Methods and systems for the treatment of HIV are disclosed, which include packaging conditional TK-SR 39 genes into virions; transducing CD34+ stem cells with the virions; transfecting the CD34+ stem cells with tat RNA; selecting modified CD34+ stem cells; injecting the modified CD34+ stem cells into a patient; and administering Ganciclovir to the patient, wherein the patient's cells infected with HIV are eliminated by the Ganciclovir. The method can comprise cloning a Zinc Finger CCR5 sequence into a Lentiviral vector expressing the tat RNA and knocking-out the CCR5 gene. Selecting the modified CD34+ stem cells is done according to flow cytometry.

### BRIEF DESCRIPTION OF THE DRAWINGS

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1A depicts steps associated with an in vitro phase of a method for eliminating HIV in accordance with a preferred embodiment;

FIG. 1B depicts steps associated with an in vivo phase of a method for eliminating HIV in accordance with a preferred embodiment;

FIG. 2 depicts charts illustrating experimental results in accordance with an example embodiment;

FIG. 3 depicts charts illustrating experimental results in accordance with an example embodiment;

FIG. 4 depicts charts illustrating experimental results in accordance with an example embodiment;

FIG. 5 depicts charts illustrating experimental results in accordance with an example embodiment;

FIG. 6 depicts charts illustrating experimental results in accordance with the an example embodiment;

FIG. 7 depicts charts illustrating experimental results in accordance with an example embodiment;

FIG. 8 depicts charts illustrating experimental results in accordance with an example embodiment;

FIG. 9 depicts charts illustrating experimental results in accordance with an example embodiment;

FIG. 10 depicts photographic images of cells in accordance with an experimental embodiment;

FIG. 11 depicts charts illustrating experimental results in accordance with an example embodiment; and

FIG. 12 depicts logical operational steps of a method for treating HIV in accordance with an alternative embodiment.

### DETAILED DESCRIPTION

The particular values and configurations discussed in these non-limiting examples can be varied and are cited merely to illustrate one or more embodiments and are not intended to limit the scope thereof.

The embodiments will now be described more fully hereinafter with reference to the accompanying drawings, in which illustrative embodiments of the invention are shown. The embodiments disclosed herein can be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. As used herein, the term “and/or” includes any and all combinations of one or more of the associated listed items.

The terminology used herein is for the purpose of describing particular embodiments only and is not intended to be limiting of the invention. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

A unique vector is disclosed herein for modification of CD34+ Hematopoietic stem cells for anti-HIV gene therapy. The vector includes a modified version of the thymidine kinase gene (SR39 mutant). This TKSR39 gene can rapidly kill cells in the presence of Ganciclovir. The disclosed vector provides three unique functions. First, the vector is expressed only in infected cells. The vector also kills the infected cells only in the presence of Ganciclovir. Finally, stem cells modified by this vector can be selected by transient expression of GFP using tat RNA transfection.

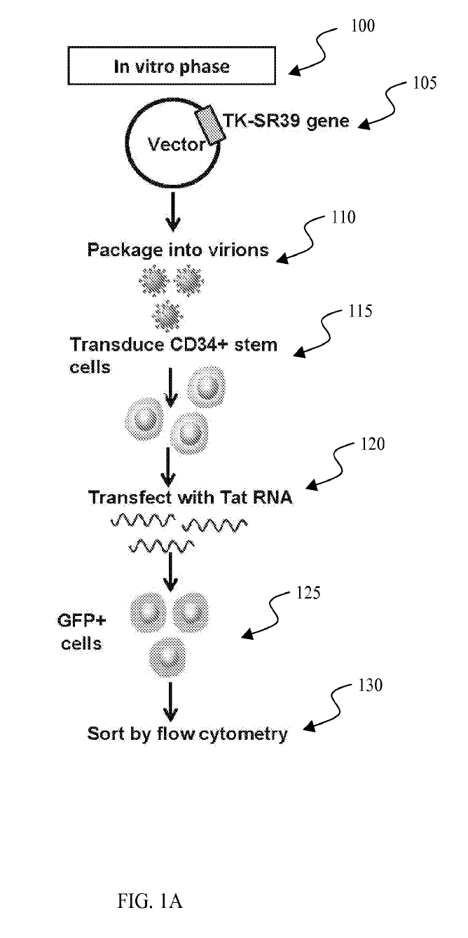

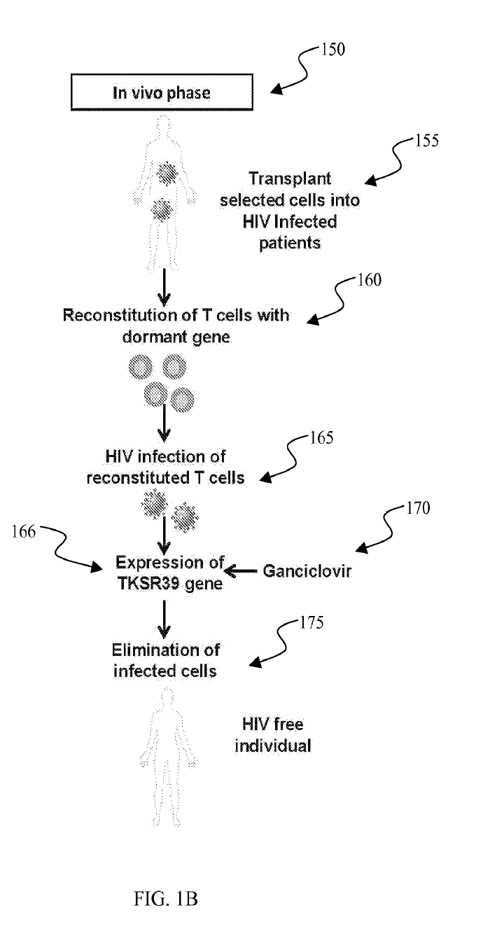

FIGS. 1A and 1B depict steps associated with a method for eliminating HIV in accordance with a preferred embodiment. The disclosed embodiments can be considered in two stages; an in vitro phase 100 and an in vivo phase 150, as illustrated in FIGS. 1A and 1B.

The embodiments disclosed herein include a mutant form of the Thymidine kinase gene referred to as SR39 (TK SR39) mutant as depicted at step 105 of FIG. 1A. This mutant is highly potent at killing cells in the presence of small amounts of Ganciclovir. Ganciclovir is a synthetic analogue of 2′-deoxy-guanosine approved for the treatment of the Herpes virus.

For safety of the vector, the TKSR39 gene can be cloned into a conditional vector. The conditional vector is preferably an HIV-derived vector that expresses the TKSR39 gene only in the presence of viral tat proteins, which are regulatory proteins that increase efficiency of viral transcription. The vector also has a tat inducible GFP gene that can be used for selection of modified stem cells.

The conditional TKSR39 vector is packaged into virus-like particles called virions as shown at step 110 of FIG. 1A. These virion particles can be used to transfer viral DNA from one cell to another. The virion particles are thus used to transduce CD34+ stem cells derived directly from the patient or a donor as shown at step 115 of FIG. 1A.

The stem cells can then be transfected with the tat RNA as depicted at step 120 of FIG. 1A to induce transient expression of Green Fluorescent Protein (GFP) in the stem cells as illustrated thereafter at step 125. The modified vector is specific and advantageous because it only expresses TKSR39 in the presence of tat. At this point, the vector is ready to be packaged and delivered to cells.

The vector inhibits the replication of virus by eliminating infected cells. The modified cells are selected using transient tat RNA transfection and GFP expression. Thus, the transient expression of GFP can be used to select the modified stem cells by flow cytometry as illustrated at step 130. By this method, only vector-modified stem cells are selected for transplantation back into the patient.

The in vivo phase of the embodiments begins as indicated at step 150 in FIG. 1B. Here, the selected vector-modified stem cells selected during the in vitro phase 100 shown in block FIG. 1A are transplanted into a patient infected with HIV as shown at step 155 of FIG. 1B. Once the cells are injected into the patient, they will produce a normal immune system, characterized by the reconstitution of T cells with the modified genes remaining dormant as shown at step 160 (in the absence of HIV tat). Once the cells are infected as depicted at step 165, they will express the TKSR39 gene as described at step 166.

At this point, the patient can be given Ganciclovir as shown at step 170. The Ganciclovir will result in elimination of the HIV infected cells as illustrated at step 175 and further suppress virus replication. This results in the elimination of HIV infected cells rending the patient HIV free in the most potent way that the human immune system controls virus replication via CD8+ cytotoxic T cells. Similar selective cytotoxic methods are reproduced to eliminate infected cells and suppress HIV replication.

Benefits of this embodiment provide targeting of the infected cells and not the virus, thus preventing development of resistance. By utilizing tat RNA to transiently express GFP in modified stem cells, only modified stem cells are selected that increase the chances of a successful transplantation. The disclosed modified vector is also safe because it does not express the gene in the absence of an HIV infection. Further, Ganciclovir has been extensively used for herpes (CMV) treatment in HIV infected patients without significant toxicity or side effects.

In another embodiment, a method for selecting TK SR39 vector modified stem cells before transplantation into patients can be implemented. Introduction of Tat DNA/RNA via transfection may suffer from drawbacks associated with its efficiency and with cell toxicity. Moreover, in primary hard to transfect cell types, like T cells and stem cells, the above limitations are even more pronounced.

Hence, in one embodiment a cloned tat gene can be introduced in a Lentiviral packaging vector and packaged into virus particles utilizing a non-integrating Lentiviral (NIL) helper construct. Using this approach, the tat gene can be delivered into cells via transduction. This overcomes both the toxicity and efficiency issues. Moreover, the cloned tat gene has a non-integrating form. Thus, the Tat-NIL particles provide transient GFP expression like that with Tat RNA.

This embodiment can be combined with steps for targeting additional genes for knock out in stem cells. In one embodiment, this is accomplished by cloning the Zinc Finger CCR5 sequence into the Lentiviral vector expressing the Tat gene. CCR5 is a co-receptor used by HIV for entry into CD4 cells and is among the most widely targeted for gene therapy approaches for HIV. Knocking down this gene is well tolerated in humans. This not only allows sorting of TK-SR39 transduced cells via GFP expression, but also causes permanent knock out of the CCR5 gene in stem cells before transplantation. Thus, the progeny of cells produced from these modified stem cells will not only bear the TK-SR39 gene, but also lack CCR5, thus preventing HIV infection and replication in modified cells via two distinct mechanisms.

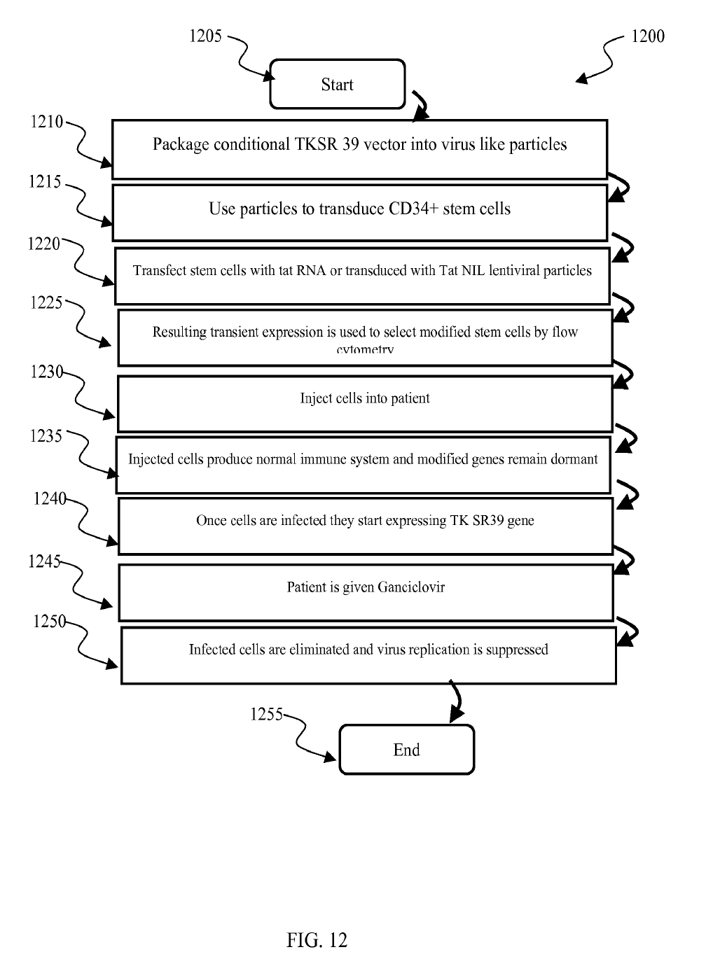

In another embodiment, as shown in FIG. 12, a method 1200 can be implemented. The method 1200 begins, as depicted at block 1205 in FIG. 12. The method 1200 can include steps such as, for example, packaging the conditional TK SR39 vector into virus like particles as indicated at block 1210. These particles can be used to transduce CD34+ stem cells derived directly from the patient or a donor as illustrated at block 1215. As depicted next at block 1220, the stem cells can be transfected with the tat RNA or transduced with Tat NIL Lentiviral particles. This results in the transient expression of GFP. The expressed GFP can be used to select the modified stem cells by flow cytometry as illustrated at block 1225.

Once these cells are injected into the patient as described at block 1230, the cells produce a normal immune system and the modified genes will remain dormant (in the absence of HIV tat) as depicted at block 1235. Once these cells become infected, the cells will start expressing TK SR39 gene as shown at block 1240.

At this point, the patient can be given Ganciclovir as illustrated at block 1245, which will result in the elimination of infected cells and will suppress virus replication as depicted then at block 1250. Elimination of infected cells is also the most potent way that the human immune system controls virus replication via CD8+ cytotoxic T cells. The method 1200 illustrated in FIG. 12 reproduces a similar selective cytotoxic process to eliminate infected cells and suppress HIV replication.

Moreover, use of zinc finger technology to knockout the CCR5 gene prevents virus infection in modified cells, which inherently lack the CCR5 co-receptor. Even if the virus evolves to switch co-receptor usage to CXCR4, the embodiments disclosed herein will still work to eliminate HIV because of the induction of toxicity via the TK-SR 39 gene. The method 1200 shown in FIG. 12 then terminates, as shown at block 1255.

Experimental data indicates that the disclosed vector is specific and only expresses TK SR39 in the presence of tat. In experiments, it has also been packaged and delivered to cells. The vector inhibits the replication of virus by eliminating infected cells. And, the modified cells have been selected in experiments using transient tat RNA transfection and GFP expression.

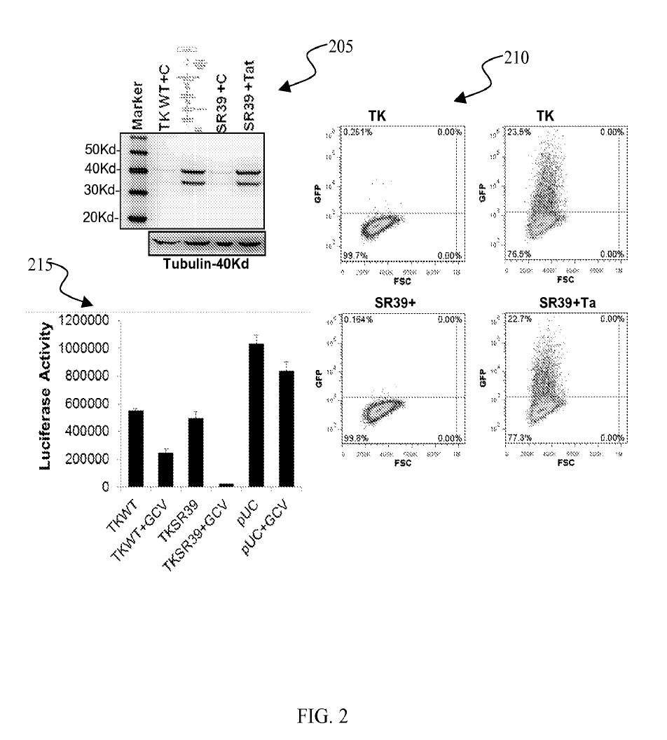

Turning now to FIG. 2, experimental results are illustrated in accordance with an example embodiment. The data depicted in FIG. 2 indicates that TK-WT genes and TK SR39 genes cloned in the conditional vector pGFPRRESA express the Thymidine Kinase (TK) gene and GFP in the presence of HIV Tat and inhibit HIV production only in the presence of Ganciclovir (GCV). HeLa cells transfected with the TKWT or TK SR39 genes expressed using the conditional vector pGFPRRESA express the TK gene as shown at graph 205 and GFP as depicted at charts 210 only in the presence of HIV Tat. The bar chart 215 illustrated in FIG. 2 indicates that the TK SR39 gene inhibits HIV production only in the presence of Ganciclovir and does so better than TK-WT.

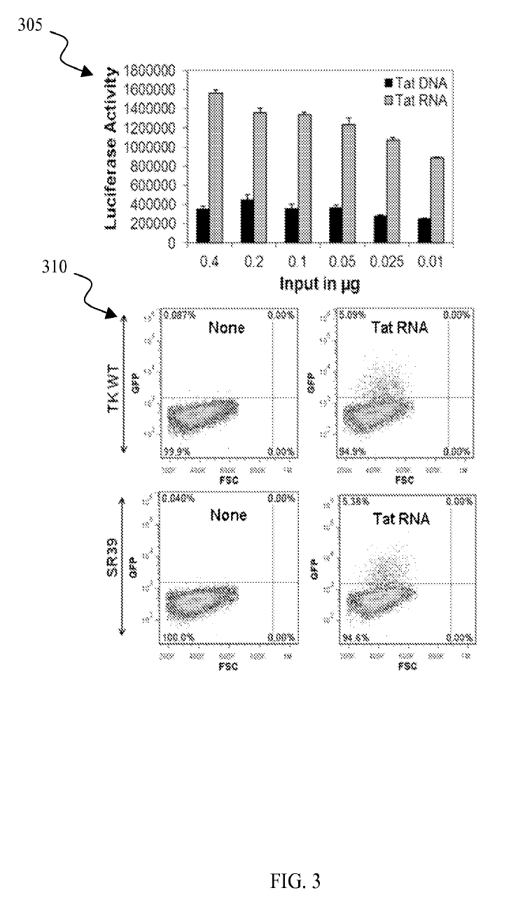

FIG. 3 depicts charts illustrating experimental results in accordance with an example embodiment. The data depicted in FIG. 3 indicates that Tat DNA or RNA can be used to sort pGFPRRESA-TK-WT or pGFPRRESA-TK-SR39 Lentiviral vector transduced cells. Graph 305 depicted in FIG. 3 depicts experimental results showing TZM-bl cells transfected with different concentrations of Tat DNA or RNA analyzed for Luciferase activity. Graph 310 shows TZM cells infected with TK-WT or TK-SR39 packaged virion particles, followed by transfection with Tat DNA or RNA. The transduced and transfected cells were then assayed for GFP expression followed by sorting of GFP positive cells in accordance with embodiments disclosed herein.

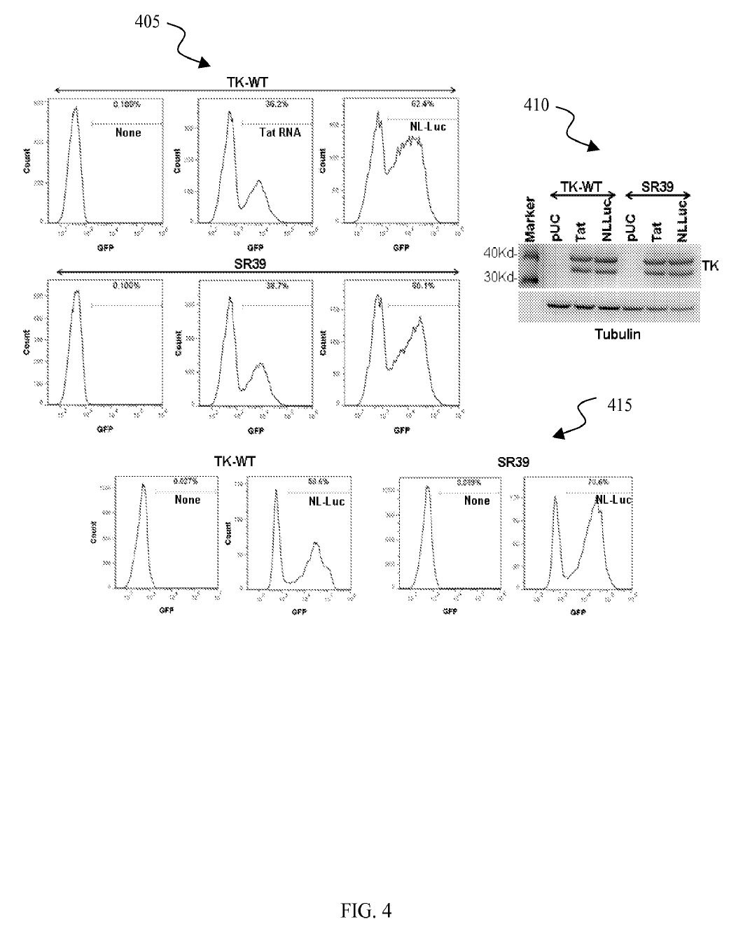

FIG. 4 depicts charts illustrating experimental results in accordance with an example embodiment. The charts in FIG. 4 indicate that sorted TZM cells maintain the dormant integrated Lentiviral vector in a stable form. Charts 405 are provided to illustrate example data indicating that GFP positive cells transduced with the TK-WT or TK-SR39 Lentiviral vector, and sorted as shown in FIG. 3, can be expanded in cell culture for prolonged periods of time. The cells can then be re-transfected with Tat DNA or RNA followed by a determination of GFP expression by flow cytometry. Chart 410 illustrates data indicative of the expression of the TK gene in control/Tat DNA transfected or NLLuc infected cells. Charts 415 illustrate the sorted cells maintained in culture for approximately 8 months (240 days) and then infected with NL-Luc to provide Rev and Tat. The TK SR39 cells maintain the TK gene in a stable form as shown by GFP expression.

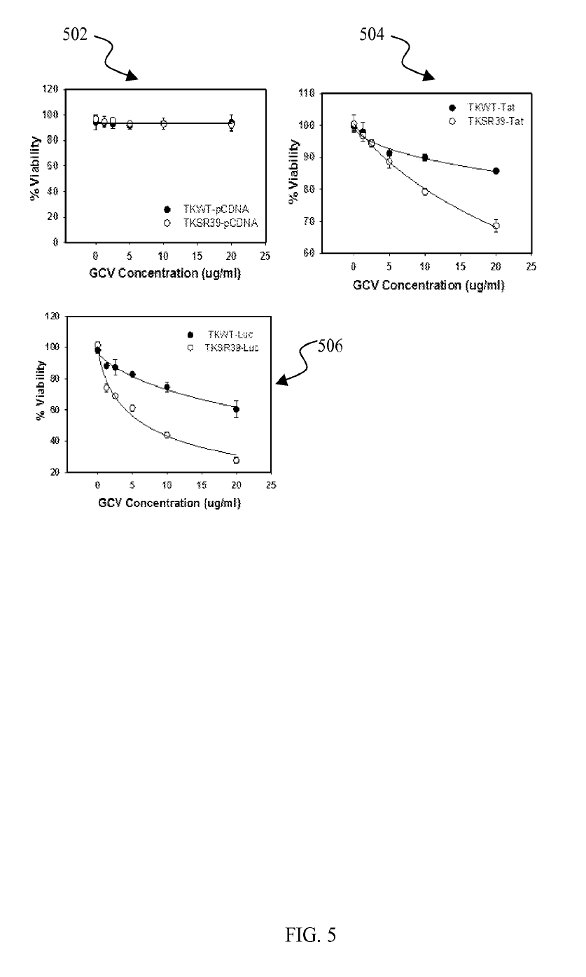

FIG. 5 depicts charts/graphs 502. 504, and 506 illustrating experimental results in accordance with an example embodiment. The charts or graphs 502, 504, 506 indicate that a Lentivirally expressed TK SR39 construct shows significantly higher cell killing in the presence of lower GCV concentrations. Sorted TZM cells transduced with the pGFPRRESA-TK-WT or pGFPRRESA-TK-SR39 vector were either transfected with Tat DNA, or infected with pNL-Luc/VSVG. GCV was added at the respective concentrations and cell viability determined 48 hours post treatment using the Cell Titer Glo viability assay.

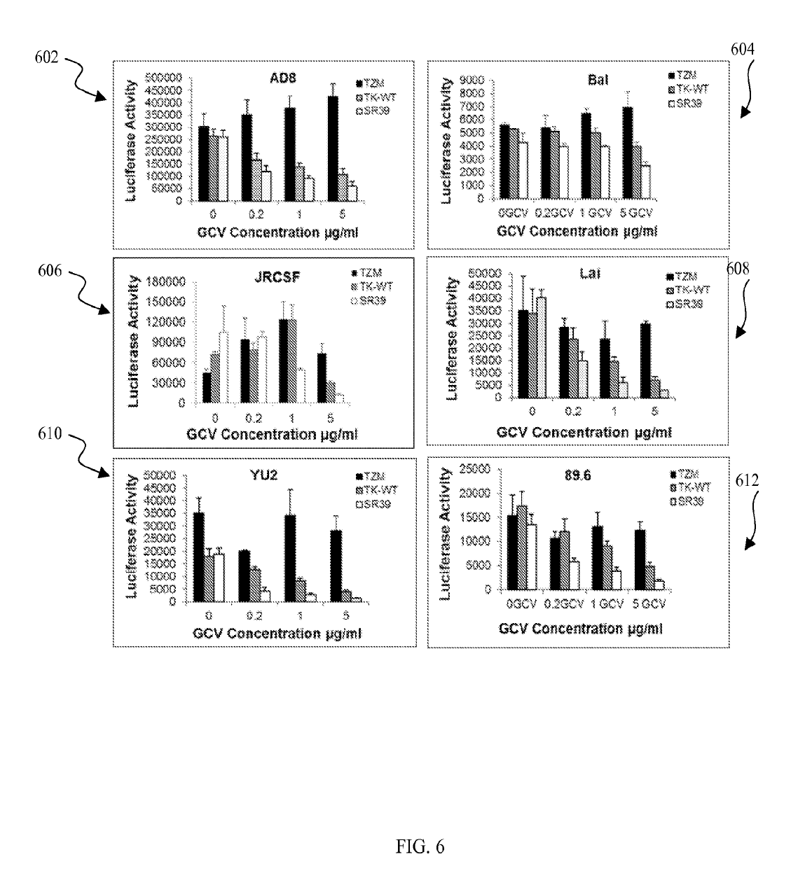

FIG. 6 depicts charts/graphs 602, 604, 606, 608, 610, and 612 illustrating experimental results in accordance with an example embodiment. The graphs 602, 604, 606, 608, 610, 612 depict data indicative of HIV inhibition in pGFPRRESA-TK-WT or pGFPRRESA-TK-SR39 transduced cells. Parental TZM cells or sorted TZM cells transduced with the pGFPRRESA-TK-WT or pGFPRRESA-TK-SR39 vector were infected with different HIV isolates. 4-5 hours post infection, culture media was replaced with RPMI containing the indicated concentrations of GCV. Culture supernatants were harvested at day 3 and 6 post infection and infectivity in TZM cells was determined.

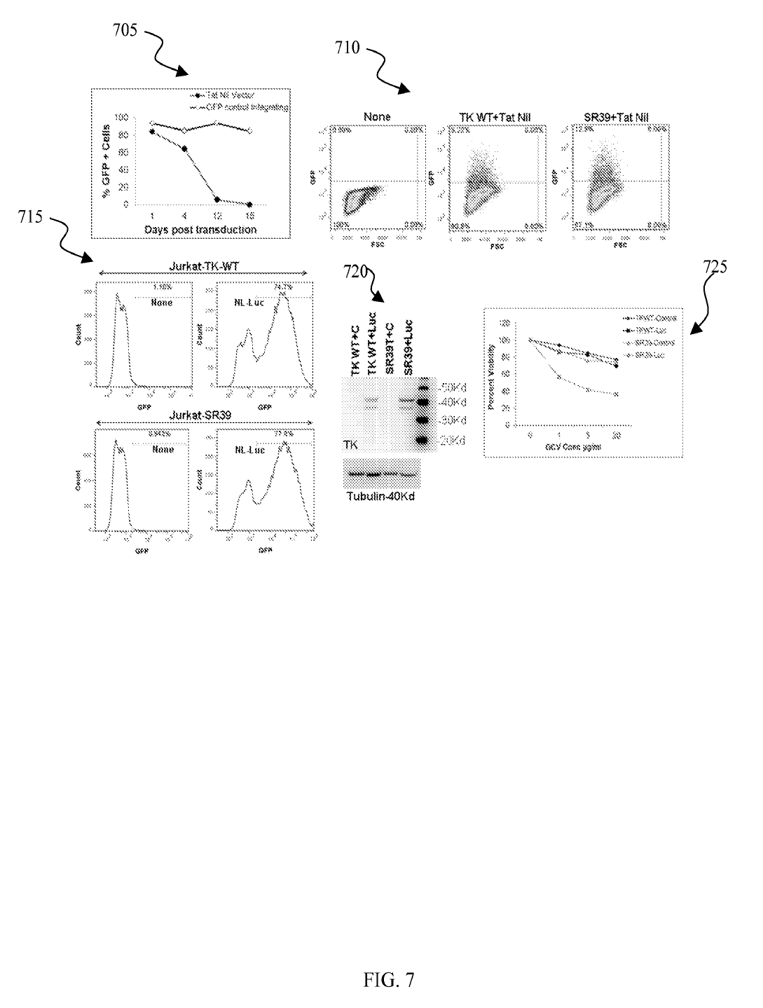

FIG. 7 depicts charts/graphs 706, 710, 715, 720, and 725 illustrating experimental results in accordance with an example embodiment. The data charted in FIG. 7 shows that the Tat NIL vector can be used to effectively sort TK WT and TK SR39 transduced cells. In particular, graph 705 illustrates that a Lentiviral construct packaged using the Tat-NIL vector expresses GFP transiently and loses GFP expression over time when compared to an integrating control. As shown at graphs 710, T cells transduced with TK SR39 followed by Tat-NIL can be sorted by GFP expression. The graphs 715 provide data indicating that T cells transduced with the TK WT and SR39 vectors lose GFP expression over time but maintain the TK gene as evidenced by the GFP expression upon infection with NL-Luc. The bar chart 720 illustrates that T cells transduced with TK WT and SR39 vectors express the TK gene only in the presence of Tat/Rev (Luc). Finally, the data shown in graph 725 indicates that SR39 transduced Jurkat cells show high cell toxicity in the presence of Tat/Rev proteins and GCV.

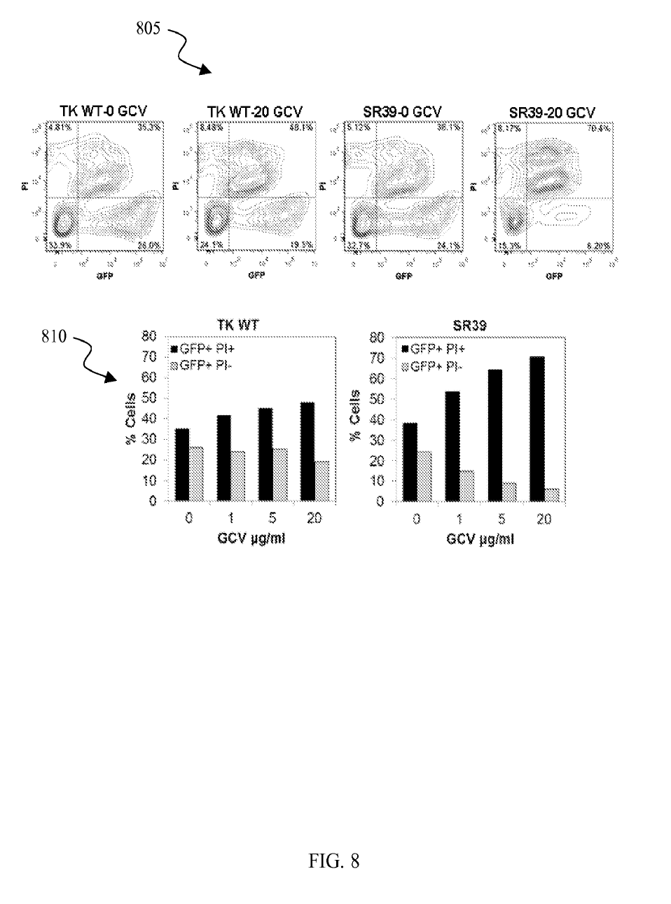

FIG. 8 depicts charts/graphs 805 and 810 illustrating experimental results in accordance with an example embodiment. The charts/graphs 805, 810 of FIG. 8 indicate that HIV infected cells undergo cell death in TK SR39 transduced cultures in the presence of GCV. As shown at graphs 805, this is made evident by the increase in GFP+PI+ cells in SR39 cultures in the presence of 20 μM GCV. Charts 810 show a percentage of each population for different GCV concentrations.

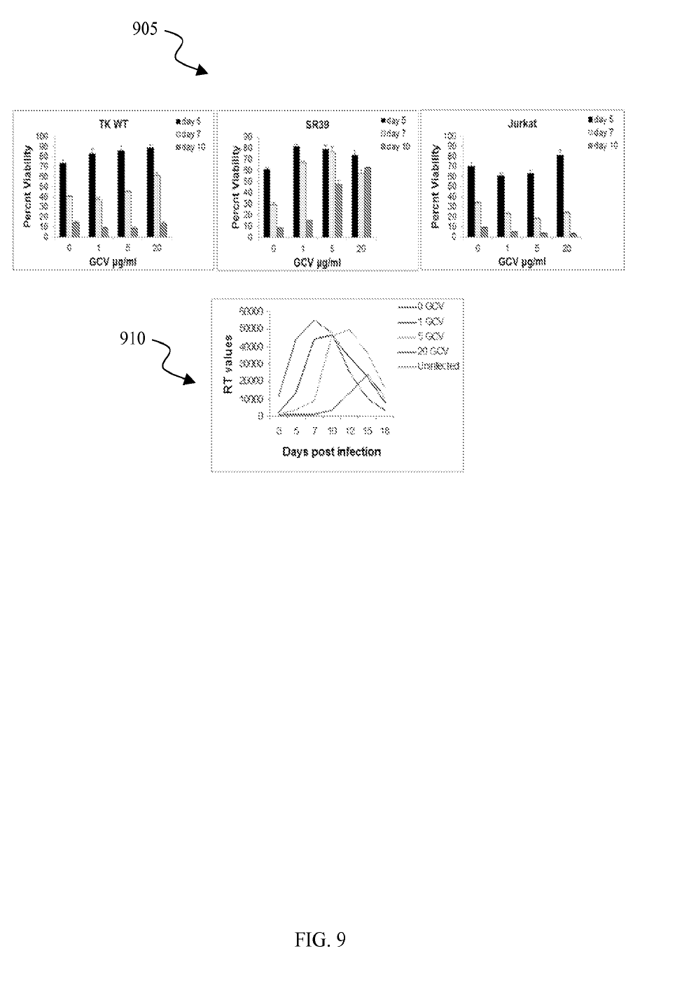

FIG. 9 depicts charts/graphs 905 and 910 illustrating experimental results in accordance with an example embodiment. Graphs 905 provide data indicating transduced T cells infected with HIV and increased viability. Graph 910 indicates diminished HIV replication in the presence of GCV.

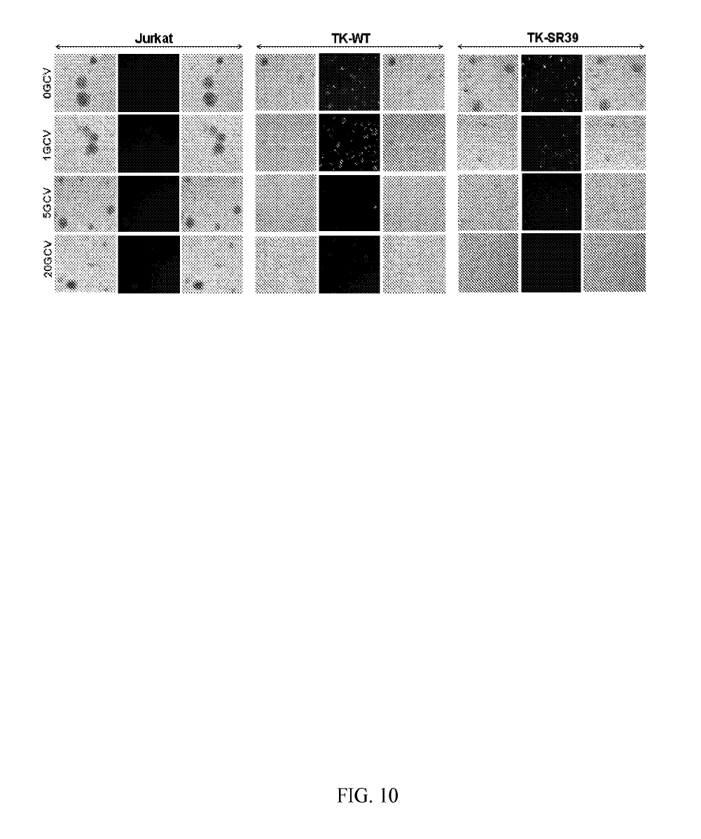

FIG. 10 depicts photographic images of cells in accordance with experimental embodiments. FIG. 10 illustrates SR39 transduced T cells infected with HIV with reduced cytopathic effect (syncytia formation) in the presence of GCV.

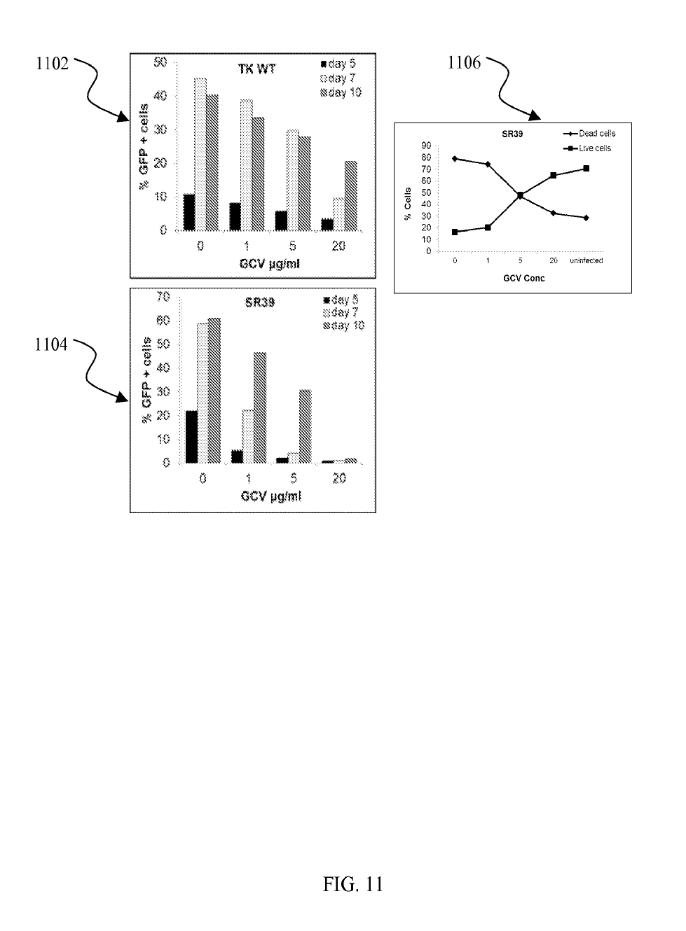

FIG. 11 depicts graphs/charts 1102, 1104, and 1106 illustrating experimental results in accordance with an example embodiment. The graphs/charts 1102, 1104, 1106 shown in FIG. 11 provide data indicating that SR39 transduced T cells infected with HIV and treated with increasing GCV concentrations evidence the elimination of infected cells accompanied by a preservation of live cell population.

The methods and systems disclosed herein uniquely provide the ability to target infected cells and not the virus, reducing the likelihood of the development of resistance. By utilizing tat RNA to transiently express GFP in modified stem cells, the methods and systems provide a means to select modified stem cells that will increase the chances of a successful transplantation. The vector disclosed herein is safe because it does not express the gene in the absence of HIV infection. Also, Ganciclovir has been extensively used for herpes (CMV) treatment in HIV infected patients without significant toxicity or side effects.

The embodiments herein thus can provide a method other than Tat RNA to sort TK SR39 vector modified stem cells. This can be accomplished by providing Tat via a non-integrating Lentiviral vector that has the added advantage of enhanced uptake in cells while limiting toxicity. Embodiments may also be included in a combination where the Zinc finger CCR5 construct is added into the Tat expressing Lentiviral vector. Targeting the CCR5 gene in stem cells is accomplished via the same Tat-NIL construct that can be used to sort modified cells (via GFP expression) and can provide additional resistance to infection by preventing virus entry in gene modified cells via CCR5 knock-out.

Based on the foregoing, it can be appreciated that a number of embodiments, preferred and alternative, are disclosed herein. For example, in one embodiment, a vector for modifying stem cells can include at least one modified thymidine kinase gene; and at least one tat gene wherein at least one stem cell modified by the vector is selected by a transient expression of GFP utilizing tat RNA transfection. In another embodiment of the vector, the tat gene can be a tat inducible GFP gene.

In another embodiment, the mutant can constitute an SR39 mutant. Such an SR39 mutant rapidly kills cells in the presence of Ganciclovir. In another embodiment, the vector may be expressed only in infected cells and kills the infected cells only in the presence of Ganciclovir. The SR39 mutant can further include TK-SR39.

In an embodiment, the vector can further include a Lentiviral packaging vector, wherein the tat gene is delivered into the stem cells via transduction. A Zinc Finger CCR5 sequence can also be implemented wherein the Zinc Finger CCR5 sequence is cloned into the Lentiviral packaging vector.

In another embodiment, a method for the treatment of HIV can include an in vitro stage composed of modifying a vector from a thymidine kinase gene, an in vivo stage comprising transplanting the modified vector into a patient, and a step or operation of providing the patient Ganciclovir, wherein the Ganciclovir eliminates HIV infected cells. In another embodiment, the thymidine kinase gene can be a TK-SR39 mutant gene.

In still another embodiment, the in vitro stage can further include expressing the modified vector in the presence of tat RNA, packaging the TK-SR39 mutant gene in a virion, transducing at least one CD34+ stem cell with the virion, and transfecting the CD34+ stem cells with the tat RNA.

In yet another embodiment, the CD 34+ stem cell can be harvested from the patient.

In another embodiment, the in vivo stage can further include packaging and delivering the modified vector to HIV-infected cells; eliminating the HIV-infected cells in a presence of Ganciclovir; and producing a normal immune system in the patient when the modified vector remains dormant in the absence of HIV tat.

In still another embodiment, a method for the treatment of HIV can be implemented, which involves packaging the conditional TK-SR 39 genes into virions; transducing CD34+ stem cells with the virions; transfecting the CD34+ stem cells with tat RNA; selecting modified CD34+ stem cells; injecting the modified CD34+ stem cells into a patient; and administering Ganciclovir to the patient, wherein the patient's cells infected with HIV are eliminated by the Ganciclovir.

In yet another embodiment, the disclosed methodology can further involve cloning a Zinc Finger CCR5 sequence into a Lentiviral vector expressing the tat RNA and knocking-out the CCR5 gene. Selecting the modified CD34+ stem cells can be accomplished according to flow cytometry.

It will be appreciated that variations of the above-disclosed and other features and functions, or alternatives thereof, may be desirably combined into many other different systems or applications. Furthermore, it can be appreciated that various presently unforeseen or unanticipated alternatives, modifications, variations or improvements therein may be subsequently made by those skilled in the art which are also intended to be encompassed by the following claims.

What is claimed is:

1. A vector for modifying stem cells, said vector comprising:
at least one modified thymidine kinase, gene; and
at least one tat gene wherein, at least one stem cell modified by said vector is selected by transient expression of GFP using tat RNA transfection.

2. The vector of claim 1 wherein said at least one tat gene comprises a tat inducible GFP gene.

3. The vector of claim 1 wherein said mutant comprises an SR39 mutant.

4. The vector of claim 3 wherein said SR39 mutant rapidly kill cells in a presence of Ganciclovir.

5. The vector of claim 4 wherein said vector is expressed only in infected cells and kills said infected cells only in said presence of Ganciclovir.

6. The vector of claim 3 wherein said SR39 mutant comprises TK SR39.

7. The vector of claim 1 further comprising:
a Lentiviral packaging vector wherein said at least one tat gene is delivered into said stem cells via transduction.

8. The vector of claim 7 further comprising:
a Zinc Finger CCR5 sequence wherein said Zinc Finger CCR5 sequence is cloned into said Lentiviral packaging vector.

9. A method for treatment of HIV, said method comprising:
an in vitro stage comprising:
modifying a vector from a thymidine kinase gene; and
an in vivo stage comprising:
transplanting said modified vector into a patient; and
providing said patient Ganciclovir wherein said Ganciclovir eliminates HIV infected cells.

10. The method of claim 9 wherein said thymidine kinase gene comprises a TK-SR39 mutant gene.

11. The method of claim 9 wherein said in vitro stage further comprises:
expressing said modified vector in a presence of tat RNA.

12. The method of claim 10 wherein said in vitro stage further comprises:
packaging said TK SR39 mutant gene in a virion.

13. The method of claim 12 wherein said in vitro stage further comprises:
transducing at least one CD34+ stem cell with said virion.

14. The method of claim 13 wherein said at least one CD 34+ stem cell is harvested from said patient.

15. The method of claim 11 wherein said in vitro stage further comprises:
transfecting said CD34+ stem cells with said tat RNA.

16. The method of claim 9 wherein said in vivo stage further comprises:
packaging and delivering said modified vector to HIV-infected cells;
eliminating said HIV-infected cells in a presence of Ganciclovir; and
producing a normal immune system in said patient when said modified vector remains dormant in an absence of HIV tat.

17. A method for treatment of HIV, said method comprising:
packaging conditional TK SR 39 genes into virions;
transducing CD34+ stem cells with said virions;
transfecting said CD34+ stem cells with tat RNA;
selecting modified CD34+ stem cells;
injecting said modified CD34+ stem cells into a patient; and
administering Ganciclovir to said patient, wherein said patient's cells infected with HIV are eliminated by said Ganciclovir.

18. The method of claim 17 further comprising:
cloning a Zinc Finger CCR5 sequence into a Lentiviral vector expressing said tat RNA.

19. The method of claim 18 further comprising:
knocking-out said CCR5 gene.

20. The method of claim 17 wherein selecting said modified CD34+ stem cells is done according to flow cytometry.
